(19) United States
(12) Patent Application Publication
(10) Pub. No.: US 2014/0031648 A1
(43) Pub. Date: Jan. 30, 2014

(54) Method and Apparatus for Conducting Blood Testing While Conserving the Blood of a Critically Ill Patient

(71) Applicant: Brandy Cross, Martinez, GA (US)

(72) Inventor: Brandy Cross, Martinez, GA (US)

(21) Appl. No.: 13/766,819

(22) Filed: Feb. 14, 2013

Related U.S. Application Data
(60) Provisional application No. 61/599,106, filed on Feb. 15, 2012.

(51) Int. Cl. A61B 5/1455; A61B 5/145; G01N 21/59

(52) U.S. Cl. CPC A61B 5/14557; G01N 21/59; A61B 5/14546
USPC 600/322; 435/288.7

(57) ABSTRACT

A method and apparatus for conducting blood tests on the blood of critically ill or anemic patients includes surrounding a transparent portion of an IV line with a sensor housing bearing radiation sources located on one side of the IV line and radiation sensors located on the opposite or other side of the IV line. A patient's blood can be drawn into the IV line, whereupon the radiation sources, which may be LEDs, are caused to pass radiation through the blood and onto the sensors. The sensors detect characteristics of the received radiation and produce a signal, which can be interpreted by a monitor to determine characteristics of the blood such as oxygen content, hemoglobin levels, and the like.

### REFERENCE TO RELATED APPLICATION

Priority is hereby claimed to the filing date of U.S. provisional patent application No. 61/599,106 filed on Feb. 15, 2012.

### TECHNICAL FIELD

This patent disclosure relates generally to medical care and more particularly to conducting blood tests on the blood of critically ill and likely anemic patients without permanently removing the blood from the patient's body while increasing the speed of results.

### BACKGROUND

Critically ill patients require reliable IV access. As peripheral IVs (the IVs you think of when going to the hospital) are in small veins, the veins can fail. When a patient's life relies on the medication they are getting in their IV, failure of the IV can be fatal. Additionally, peripheral IVs generally are only left in place by medical personnel for a max of 3 days. Peripheral IVs deliver fluids to small veins. As these veins carry relatively small amounts of blood at a time, medical personnel are limited by the types of medications and concentrations of both medications and nutrition that are introduced to a patient's body through these veins.

A central IV, often incorporating a triple lumen catheter, is a large IV. It includes a long catheter that is placed in a major vein usually subclavian (beneath the collar bone), internal jugular (in the neck) or femoral (in the groin). Central IVs are used to deliver chemotherapy, complete IV nutrition, IV fluids, IV medications to keep blood pressure up for a patient in shock, and a variety of other treatments. In addition, certain types of monitoring can be done through a central IV that cannot be done through a peripheral IV, including monitoring the CVP (central venous pressure) in the large veins of the body. Monitoring the CVP can be used to determine if a patient's blood pressure is low because they need more IV fluid or due to another cause such as heart failure. Medical personnel also are able to draw blood through the lines of a central IV as central IVs are larger and do not tend to break-up red blood cells as can occur when drawing blood through a peripheral IV.

Critically ill and emergency patients often will have lost significant volumes of blood from surgery or trauma. Additionally, their bodies realize that there is a stressor. Whether the stressor is trauma or infection, the body reacts in a similar way; that is to, in essence, “hide” iron from bacteria, which thrive on iron. As such, the iron stores are kept safe by the body, and few red blood cells are produced. To add insult to injury, physicians treating these patients need to determine multiple blood characteristics and indicators to care for critically ill patients. These range from hourly glucose level indicators for patients on insulin drips to checking hemoglobin every 6 hours for a patient with an injured spleen. To conduct such tests, blood generally is drawn from the patients and sent to a medical laboratory for testing. The blood that is drawn out is often not replaced by the body in a timely fashion, particularly in critically ill patients. Thus, patients can become more anemic due to the gradual loss of blood that is withdrawn for testing. Additionally, labs are expensive to run and risk the health of nurses and other staff due to the exposure to sharp needles and patient blood. Results from these labs often can be delayed an hour or longer due to the amount of time required to carry the blood physically to the lab, operate the various machines to determine results, and post the results for medical personnel to view.

Pulse oximetry is used often in an intensive care unit (ICU) and elsewhere. Commercially available pulse oximeters determine certain blood characteristics by projecting light of selected wavelengths through a patient's fingernail bed. The patient does not feel it, but based on the spectral absorption patterns of the light and the dynamics of those patterns (how they change over time), it is possible to detect how much hemoglobin a patient has, what percentage of the red blood cells are carrying oxygen, and other characteristics, without drawing blood from a patient. While this technique is widely useful, problems include that the characteristics of the light and its absorption can be altered or occulted by nail polish, dark nail beds, and vasoconstriction, which can be experienced by, for instance, a patient in shock or a dehydrated patient. Such situations, which are not uncommon, can result in inaccurate and/or unusable results garnered through pulse oximetry. Nevertheless, the medical device industry is very close to being able to detect additional characteristics of blood such as glucose levels, through non-invasive optical absorption measurement techniques, but accuracy and reliability are still being improved.

There is therefore a need for a method and apparatus with which doctors and medical personnel can conduct non-invasive reliable blood testing on the blood of a critically ill or otherwise anemic patient without permanently removing blood from the patient's body. It is to the provision of such a method and apparatus that the invention disclosed herein is primarily directed.

### SUMMARY

U.S. provisional patent application No. 61/599,106, to which priority is claimed above, is hereby incorporated by reference as if fully set forth herein. Briefly described, a central IV preferably includes a catheter and multiple lines through which fluid and medication can be delivered through the catheter to a patient's bloodstream. At least a portion of one of the lines is transparent and a section of this transparent portion is surrounded by a sensor housing. A radiation source or multiple radiation sources such as an array of light emitting diodes (LEDs) is arranged in the sensor housing on one side of the clear line. One or more radiation sensors are arranged in the sensor housing on an opposite side of the clear line. The radiation source may emit visible light, infrared light, ultraviolet light, or radiation of other wavelengths, or combinations thereof. The sensors on the opposite side of the clear line receive radiation from the sensors after the radiation has passed through the clear line and can measure characteristics of the radiation as may be affected by substances within the clear line through which the radiation passes.

In use, medical personnel cause a specimen of a patient's blood to be drawn into the clear line until the blood is located within the sensor housing. Measurements of various characteristics of the blood specimen can then be made by projection radiation through the blood on one side of the clear line and measuring the resulting characteristics of the light emerging from the blood on the other side of the clear line. Then, when the measurements have been completed, medical personnel can simply flush the patient's blood back into his or her body by introducing a saline solution through the clear line of the main catheter. It will thus be seen that blood testing can be done as often as desired without removing valuable blood from the bloodstream of a patient. This can make a big difference in recovery prognoses, particularly for critically ill or anemic patients who need to retain all of their natural blood that they can.

It will thus be seen that an improved method and apparatus is now provided that addresses the above mentioned issues and more, that allows blood testing as frequently as desired without jeopardizing a patient's blood supply, and that does not expose medical personnel to the blood of patients. These and other aspects, features, and advantages of the present invention will become more apparent upon review of the detailed description set forth below taken in conjunction with the accompanying drawing figures, which are briefly described as follows.

### BRIEF DESCRIPTION OF THE DRAWINGS

FIG. 1 is a drawing showing an apparatus according to one embodiment of this invention attached to a patient.

FIG. 2 is a side elevational view of a sensor housing containing radiation sources and corresponding radiation sensors according to aspects of the invention.

FIG. 3 is a photograph of a triple lumen catheter of the type with which the sensor module of the current invention might be used.

### DETAILED DESCRIPTION

Referring now in more detail to the drawing figures, wherein like reference numbers indicate like parts throughout the several views, FIG. 1 illustrates a patient P into which a main catheter 12 has been inserted. Three lines L1, L2, and L3 are coupled to and in communication with the catheter. Two of the lines L1 and L2 in FIG. 1 are shown not in use and the third or central line L3 is shown connected to an external source of IV fluids. Most, if not all, central lines L3 of multi-lumen catheters 12 have a section 13 of the line that is a clear transparent pliable plastic. The majority of time, doctors and medical personnel deliver a clear crystalloid solution of IV fluid through the central line to the patient. However, the blood of a patient P also can be drawn from his or her body into the clear portion of the central line if desired by, for example, reversing the flow through the central line L3. The traditional uses of multi lumen catheters are well known to doctors and other medical personnel.

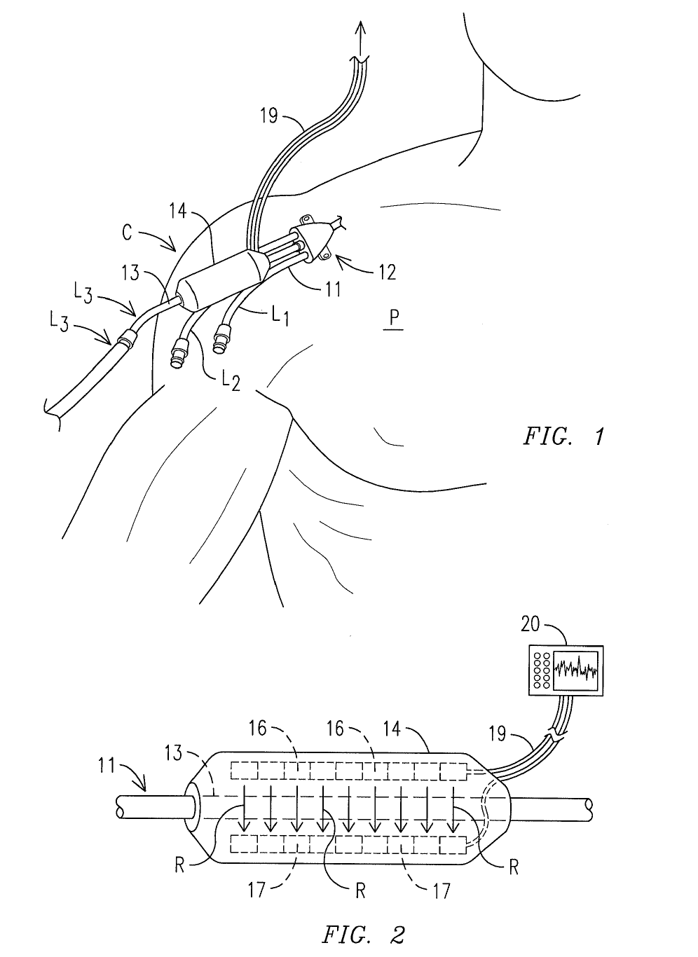

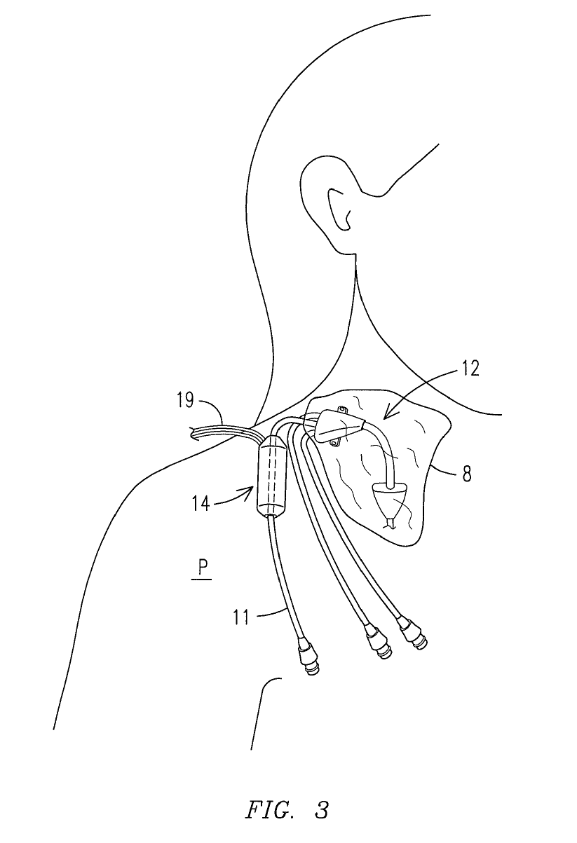

According to the present invention, a generally cylindrical sensor housing 14 is configured to be placed around the clear section 13 of the central line L3. The sensor housing 14 contains LEDs 16 (FIG. 2) or other sources of electromagnetic radiation on the superior aspect of the cylindrical sensor housing. In the embodiment of FIG. 2, an array of LEDs 16 is arranged along a length of the central line and each LED may emit radiation of a different frequency or of different spectral characteristics for performing different tests. A corresponding array of optical sensors 17 is disposed on the inferior aspect of the sensor housing 14 and also extends along a length of the clear portion 13 of the main line 11. In the illustrated embodiment, each sensor is aligned with a corresponding LED on the opposite side of the main line L3. Electrical leads 19 connect the array of LEDs and their corresponding sensors to remote equipment 20 that operates them in known ways.

The LEDs 16 are arranged to project electromagnetic radiation such as visible light, infrared radiation, ultraviolet radiation, or radiation in other wavelengths, through the clear section 13 of the central line 11 that is surrounded by the sensor housing, as indicated by arrows R. Each of the sensors 17 is arranged and positioned so that the radiation from its corresponding LED that passes through the clear portion 13 of the central line L3 impinges on the sensor. Baffles (not shown) may be disposed between each LED-Sensor pair to isolate the pair from radiation from the other LEDs of the array or, alternatively, electronic filters may be used for each sensor to reject wavelengths outside those of the corresponding LED source.

It will be seen that with the above construction, radiation projected from each of the LEDs passes through any fluid contained within the clear portion 13 of the main line 11. The fluid, on the other hand, may absorb parts of the radiation, scatter parts of the radiation, reflect parts of the radiation, and the like. Accordingly, the spectral characteristics of the radiation reaching each sensor are changed as a function of the optical properties of the fluid within the main line 11. For instance, if human blood is the substance in the clear section 13, then the sensors may detect the quantity of radiation absorbed by the blood and at what wavelengths the absorption occurs as well as temporal variations in these and other parameters. Such characteristics have been found to be indicative of certain properties of the blood such as, for instance, oxygen levels, hemoglobin levels, and other blood characteristics.

It will thus be seen that a variety of blood tests can be conducted optically while a patient's blood is drawn into and contained within the clear portion 13 of the main line L3. When the blood tests are complete and the results tabulated, the blood used for the tests can be re-introduced into a patient's body by again reversing the flow of fluid through the catheter. Thus, the patient has not suffered any permanent loss of blood, which can be very important for anemic patients or critically ill patients. Further, the blood tests can be conducted again and again as frequently as desired without resulting in permanent blood loss for the patient. Finally, since the blood does not go to a lab, it is not handled by medical personal and thus saves time and money, and prevents potentially dangerous exposure.

In use, the sensor housing 14 with its internal LEDs 16 and sensors 17 is mounted to a clear plastic portion 13 of the central line L3 as illustrated in FIG. 1. For example, the sensor housing may be made available in a clamshell configuration that can be closed around the central line and snapped shut. This positions the LEDs and sensors appropriately on opposing sides of the central line. Appropriate electrical connections and data lines 19 can be coupled to the housing 14 to power the LEDs 16 and transmit signals from the sensors 17. The signals are transmitted to a monitor 20 or other auxiliary equipment for interpreting the signals to extract blood characteristics from the signals; or, in other words, to “read” the sensors. In one embodiment, the sensors 17 can be “white balanced” and/or otherwise calibrated when clear fluids are running through the transparent portion of the main line with radiation from the LEDs 16 is passing through the clear fluid. In this way, contributions to the signals (i.e. variations in the transmitted radiation) produced by the sensors as a result of the IV fluid are accounted for.

Once the sensors 17 are white balanced and calibrated, blood from the patient P is drawn into the clear section 13 of the central line 11, just as one would do to withdraw blood for labs. However, the blood is not extracted from the patient, but rather drawn only enough so that undiluted blood is disposed within the clear section 13 of the central line L3. The series of LEDs 16 are activated by the remote monitoring hardware 20 to pass radiation through the blood in the clear section 13 and onto the sensors 17 on the other side of the clear portion. Each LED may be configured to produce radiation of a selected wavelength and/or a selected bandwidth or spectral characteristics for detection of a corresponding element in the blood according to known oximetry-like techniques. Alternatively, the LEDs may all produce the same radiation to be detected by all of the corresponding sensors for enhancing detection of a particular element within the blood. Generally, the LEDs and sensors may be activated anywhere in between these extremes as conditions dictate.

The sensors 17 may detect spectral absorption patterns resulting from corresponding elements in the blood, attenuation of the radiation, and other characteristics of the radiation that has passed through the blood. This information is sent directly to a bedside monitor 20, which calculates and displays the needed blood characteristics. Accordingly, a barrage of blood tests can be conducted while a patient's blood is contained within the clear portion 13 of the main line L3.

With the blood tests having been completed, an attending nurse or other medical personnel simply flushes the tested blood back into the patient P with IV fluids. The physicians have their values the patient lost no blood, and nursing staff is always safely separated from any contact with the blood. Further, the sensor housing 14 and its LEDs 16 and sensors 17 are isolated from the patient's blood and therefore may be reused after an appropriate disinfection and cleaning and so the method of this invention is likely less expensive than traditional lab tests. Additionally, the blood characteristics are available almost instantaneously as opposed to waiting for the blood to get physically to the lab and the lab tests to be run, which often can take an hour or more. For very critical patients who require continuous testing, it is possible according to the invention to circulate a patient's blood with a small pump that constantly removes it through one catheter, moves it through the testing region, and returns it through another catheter back into a patient's blood system. In this way, continuous, real time, second by second monitoring can be conducted; however, second by second monitoring may not be needed in all cases.

FIG. 3 simply shows a triple lumen catheter 12 inserted into the bloodstream of a patient P and secured to the patient's skin with tape 8. One of the lines of the catheter has a sensor housing 14 according to the invention attached to a transparent portion thereof for conducting blood tests as described.

The invention has been described herein in terms of preferred exemplary embodiments considered by the inventor to represent the best modes of carrying out the invention. The invention is not limited to the exemplary embodiments, however, and a wide variety of additions, deletions, and modifications, both subtle and gross, might well be made to the illustrated embodiments by those of skill in the art without departing from the spirit and scope of the invention.

What is claimed is:

1. An apparatus for conducting blood testing without permanently removing blood from a patient comprising:
a catheter for insertion into the bloodstream of a patient;
at least one line communicating with the catheter and having a transparent portion that is substantially transparent to radiation of predetermined characteristics;
at least one radiation source arranged to projection radiation toward the transparent portion of the line;
at least one radiation sensor arranged to receive radiation from the radiation source after the radiation has passed through the transparent portion of the line, the radiation sensor producing a signal in response to the received radiation; and
a monitor connected to the at least one radiation sensor for receiving the signal and calculating a characteristic of a substance contained within the substantially transparent portion of the line based upon characteristics of the signal.

2. The apparatus of claim 1 wherein the at least one radiation source comprises a plurality of radiation sources.

3. The apparatus of claim 2 wherein at least some of the plurality of radiation sources project radiation that is different in characteristics from radiation produced by others of the radiation sources.

4. The apparatus of claim 3 wherein the characteristics comprise spectral bandwidth.

5. The apparatus of claim 3 wherein the characteristics comprise wavelength.

6. The apparatus of claim 1 further comprising a sensor housing containing the at least one radiation source, the at least one radiation sensor, and at least part of the transparent portion of the line.

7. The apparatus of claim 6 wherein the sensor housing is configured to be connected to the transparent portion of the line and removed from the transparent portion of the line.

8. The apparatus of claim 6 wherein the sensor housing completely contains the at least one radiation source, the at least one radiation sensor, and the at least part of the transparent portion of the line.

9. The apparatus of claim 1 wherein the signal is characteristic of human blood contained within the transparent portion of the line and the monitor calculates a property of the human blood based upon the signal.

10. A method of conducting blood testing without permanently removing blood from a patient comprising the steps of:
(a) inserting a catheter into the patient in such a way that the catheter communicates with the bloodstream of the patient, the catheter having at least one line with a transparent portion communicating therewith;
(b) disposing a source of radiation to project radiation onto the transparent portion of the at least one line;
(c) disposing a radiation sensor to receive radiation projected by the sensor after the radiation has passed through the transparent portion of the at least one line.
(d) drawing blood from the patient's bloodstream into the transparent portion of the at least one line;
(e) projecting radiation with the source of radiation toward the blood in the transparent portion of the at least one line;
(f) receiving a signal from the radiation sensor characteristic of a property of the blood in the transparent portion of the at least one line through which the radiation has passed;
(g) calculating a property of the blood based upon the received signal; and
(h) returning the blood within the transparent portion of the at least one line back to the patient's bloodstream.

11. The method of claim 10 wherein step (b) comprises disposing a plurality of sources of radiation to projecting radiation onto the transparent portion of the at least one line.

12. The method of claim 11 wherein step (c) comprises disposing a plurality of radiation sensors to receive radiation projected by the plurality of sources of radiation after the radiation has passed through the transparent portion of the line.

13. The method of claim 12 further comprising causing some of the sources of radiation to project radiation of different characteristics than others of the sources of radiation.

14. The method of claim 10 wherein steps (b) and (c) comprise removably attaching a sensor housing around the transparent portion of the at least one line, the sensor housing containing the source of radiation and the radiation sensor.

15. The method of claim 14 wherein the sensor housing may be sterilized and re-used when removed.

16. The method of claim 10 wherein steps (d) and (h) comprise inserting a second catheter into the patient in such a way that the second catheter communicates with the bloodstream of the patient and circulating the patient's blood from the catheter, through the transparent portion of the line, and back to the patient's bloodstream through the second catheter.

17. A method of conducting blood tests on a patient's blood comprising drawing the patient's blood into a lumen having walls that are at least partially transparent to radiation, projecting radiation through the blood within the lumen, detecting characteristics of the radiation after it has passed through the blood, calculating a property of the blood based upon the detected characteristics, and returning the drawn blood back to the patient.
